(19) United States
(12) Patent Application Publication
(10) Pub. No.: US 2023/0000783 A1
(43) Pub. Date: Jan. 5, 2023

(54) DUAL SUPRAMOLECULAR NANOPARTICLE VECTORS ENABLE CRISPR/CAS9-MEDIATED KNOCKIN OF RETINOSCHISIN 1 GENE-A POTENTIAL NON-VIRAL THERAPEUTIC SOLUTIONS FOR X-LINKED JUVENILE RETINOSCHISIS

(71) Applicants: The Regents of the University of California, Oakland, CA (US); National Yang-Ming University, Taipei (TW)

(72) Inventors: Hsian-Rong Tseng, Oakland, CA (US); Shih-Hwa Chiou, Taipei (TW); Peng Yang, Oakland, CA (US); Shih-Jie Chou, Taipei (TW)

(73) Assignees: The Regents of the University of California, Oakland, CA (US); National Yang-Ming University, Taipei (TW)

(21) Appl. No.: 17/776,989

(22) PCT Filed: Nov. 13, 2020
(86) PCT No.: PCT/US2020/060536

§ 371 Date: May 13, 2022

Related U.S. Application Data
(60) Provisional application No. 62/935,886, filed on Nov. 15, 2019.

(51) Int. Cl. A61K 9/51; C12N 15/113; C12N 15/11; C12N 15/85; C12N 15/90; C12N 9/22; A61P 27/02

(52) U.S. Cl. CPC A61K 9/5146; C12N 15/113; C12N 15/111; C12N 15/85; C12N 15/90; C12N 9/22; A61P 27/02; A61K 48/00

(57) ABSTRACT

Compositions, systems and methods for delivering CRISPR/Cas9-based genome editing system and a donor protein to a cell.

### CROSS-REFERENCE OF RELATED APPLICATION

This application claims priority to U.S. Provisional Application No. 62/935,886 filed Nov. 15, 2019; the entire contents of all of which are hereby incorporated by reference.

### BACKGROUND

### 1. Field of the Invention

The field of the currently claimed embodiments of this invention relates to compositions, systems and methods for delivering CRISPR/Cas9-based genome editing system and a donor protein to a cell.

### 2. Discussion of Related Art

The clustered regularly interspaced short palindromic repeats, CRISPR-associated protein 9 (CRISPR/Cas9) system is revolutionizing gene therapy.^{[1]} A CRISPR/Cas9-mediated gene editing system is composed of two functional components, i.e., Cas9 endonuclease and an engineered short, single-guide RNA (sgRNA), which form a ribonucleoprotein complex, Cas9.sgRNA. Based on a base-pairing mechanism, Cas9.sgRNA complex recognizes and cuts the targeted site, precisely inducing a double-strand break (DSB).^{[1]} Subsequently, endogenous DNA repair occurs via either a non-homologous end joining (NHEJ) or a homology directed repair (HDR) pathway. As a general therapeutic solution for treating genetic diseases,^{[2]} the HDR pathway is often adopted for CRISPR/Cas9-mediated knockin,^{[3]} by which a therapeutic gene carried by donor DNA (dDNA) is integrated into DSB. However, HDR-based CRISPR/Cas9-mediated knockin is less efficient in vivo since the HDR pathway is not readily accessible to non-dividing cells in tissue.^{[4]} By contrast, the NHEJ pathway is active in both non-dividing and proliferating cells, and thus, can be adopted to achieve more efficient in vivo CRISPR/Cas9-mediated knockin. A homology-independent targeted integration (HITI) strategy^{[5]} was developed based on the NHEJ pathway to enable robust knockin of non-dividing cells in vivo. In brief, HITI strategy introduces two pre-determined CRISPR/Cas9 target sites into dDNA. After cutting the targeted sites present in both genomic DNA and dDNA, the resulting three DSB sites undergoes endogenous DNA repair via the NHEJ pathway to achieve gene integration.^{[5]} In animal studies, the HITI strategy enables more efficient CRISPR/Cas9-mediated knockin, suitable for a local gene-editing solution in organs, like eyes and brains.^{[5a]} However, the technical challenge remains to develop safe and reliable delivery vectors that can co-deliver Cas9.sgRNA and dDNA into the target cells and/or tissues.

X-linked juvenile retinoschisis, XLRS is a condition characterized by impaired vision that begins in childhood in males. Approximately 200 mutations of the RS1 gene have been identified as associated with either decreases in or complete loss of functional retinoschisin, which disrupts the maintenance and organization of cells in the retina.^{[6]} Recently, a small number of clinical trials^{[7]} were initiated to evaluate the safety and efficacy of adeno-associated virus (AAV)-based gene therapy approaches, which introduce functional retinoschisin in retina to treat XLRS. To date, none of the AAV-based RS1 gene therapy approaches^{[8]} has reached a satisfactory clinical endpoint. Meanwhile, researchers have been exploring CRISPR/Cas9-mediated knockin of RS1 gene to achieve a curative therapeutic solution for XLRS. While AAV^{[9]} is also frequently used for delivering CRISPR/Cas9 system, its limited packaging capacity (<4.7 kb)^{[10]} and safety concerns on viral integration and immunogenicity remain. Alternative approaches harness the potential of non-viral vectors,^{[11]} including lipids,^{[12]} polymers^{[13]} and nanoparticles^{[14]} for carrying Cas9, guide RNA (gRNA), and donor DNA (dDNA). Although there has been substantial progress made for CRISPR/Cas9-mediated gene knockout,^{[14c, 15]} and knockdown^{[16]} using non-viral vectors along the NHEJ pathway, relatively limited results have been obtained for CRISPR/Cas9-mediated knockin,^{[14d, 17]} especially for integration of a full-length gene.

### INCORPORATION BY REFERENCE

All publications and patent applications identified herein are incorporated by reference in their entirety and to the same extent as if each individual publication or patent application was specifically and individually indicated to be incorporated by reference.

### SUMMARY

An embodiment of the invention relates to a composition for delivering a nucleic acid encoding an endonuclease and a nucleic acid sequence encoding a donor protein to a cell including: a first plurality of self-assembled supramolecular nanoparticles (SMNPs), each of the first plurality of self-assembled supramolecular nanoparticles (SMNPs) including: a plurality of binding components, each having a plurality of binding regions; a plurality of cores that are suitable to at least provide some mechanical structure to the plurality of self-assembled supramolecular nanoparticles (SMNPs), the plurality of cores including at least one core binding element adapted to bind to the binding regions to form a first inclusion complex, wherein the plurality of binding components and the plurality of cores self-assemble when brought into contact to form the first plurality of self-assembled supramolecular nanoparticles (SMNPs); a plurality of terminating components, each having a single terminating binding element that binds to remaining binding regions of one of said plurality of binding components by forming a second inclusion complex, wherein the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle; the nucleic acid sequence encoding the endonuclease; and a nucleotide sequence including a recognition sequence specific to the endonuclease; and a second plurality of self-assembled supramolecular nanoparticles (SMNPs), each of the second plurality of self-assembled supramolecular nanoparticles (SMNPs) including: a plurality of binding components, each having a plurality of binding regions; a plurality of cores that are suitable to at least provide some mechanical structure to the plurality of self-assembled supramolecular nanoparticles (SMNPs), the plurality of cores including at least one core binding element adapted to bind to the binding regions to form a first inclusion complex, wherein the plurality of binding components and the plurality of cores self-assemble when brought into contact to form the second plurality of self-assembled supramolecular nanoparticles (SMNPs); a plurality of terminating components, each having a single terminating binding element that binds to remaining binding regions of one of said plurality of binding components by forming a second inclusion complex, wherein the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle; and the nucleic acid sequence encoding the donor protein. In such an embodiment, the nucleic acid sequence encoding the endonuclease and the nucleotide sequence including a recognition sequence specific to the endonuclease are encapsulated within each of the first plurality of self-assembled SMNPs, and the nucleic acid sequence encoding the donor protein is encapsulated within each of the second plurality of self-assembled SMNPs.

An embodiment of the invention relates to a method for delivering a nucleic acid encoding an endonuclease and a nucleic acid sequence encoding a donor protein to a cell including: providing a first plurality of self-assembled supramolecular nanoparticles (SMNPs); providing a second plurality of self-assembled SMNPs; and contacting the cell with at least one of the first plurality of self-assembled SMNPs and with at least one of the second plurality of self-assembled SMNPs, such that the at least one of the first plurality of self-assembled SMNPs and the at least one of the second plurality of self-assembled SMNPs are each taken up by the cell. In such an embodiment, each of the first plurality of self-assembled supramolecular nanoparticles SMNPs includes: a plurality of binding components, each having a plurality of binding regions; a plurality of cores that are suitable to at least provide some mechanical structure to the plurality of self-assembled SMNPs, the plurality of cores including at least one core binding element adapted to bind to the binding regions to form a first inclusion complex, wherein the plurality of binding components and the plurality of cores self-assemble when brought into contact to form the first plurality of self-assembled SMNPs; a plurality of terminating components, each having a single terminating binding element that binds to remaining binding regions of one of said plurality of binding components by forming a second inclusion complex, wherein the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle; the nucleic acid sequence encoding the endonuclease; and a nucleotide sequence including a recognition sequence specific to the endonuclease. In such an embodiment, each of the second plurality of self-assembled SMNPs includes: a plurality of binding components, each having a plurality of binding regions; a plurality of cores that are suitable to at least provide some mechanical structure to the plurality of self-assembled SMNPs, the plurality of cores including at least one core binding element adapted to bind to the binding regions to form a first inclusion complex, wherein the plurality of binding components and the plurality of cores self-assemble when brought into contact to form the second plurality of self-assembled SMNPs; a plurality of terminating components, each having a single terminating binding element that binds to remaining binding regions of one of said plurality of binding components by forming a second inclusion complex, wherein the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle; and the nucleic acid sequence encoding the donor protein. In such an embodiment, the nucleic acid sequence encoding the endonuclease and the nucleotide sequence including a recognition sequence specific to the endonuclease are encapsulated within each of the first plurality of self-assembled SMNPs, and the nucleic acid sequence encoding the donor protein is encapsulated within each of the second plurality of self-assembled SMNPs.

### BRIEF DESCRIPTION OF THE DRAWINGS

Further objectives and advantages will become apparent from a consideration of the description, drawings, and examples.

FIGS. 1A-1C are illustrations showing example embodiments according to the invention;

FIGS. 2A-2C are illustrations and eletrophoretograms showing optimization of self-assembling supramolecular nanoparticles (SMNPs) for delivering Cas9 according to an embodiment of the invention;

FIGS. 3A-3C are illustrations, fluorescent images and a data chart showing optimization of self-assembling supramolecular nanoparticles (SMNPs) for delivering a donor gene plasmid according to an embodiment of the invention;

FIGS. 4A-4C are illustrations, electron microscopy images, and data graphs showing optimization of SMNPs encapsulating a Cas9/sgRNA plasmid and a donor gene plasmid according to an embodiment of the invention;

FIGS. 5A-5F are illustrations, fluorescent micrographs, and data graphs showing results of experiments assaying the delivery of a Cas9/sgRNA plasmid and a donor gene plasmid to cells in vitro using SMNPs according to an embodiment of the invention; and

FIGS. 6A-6E are illustrations, fluorescent micrographs, and data graphs showing results of experiments assaying the delivery of a Cas9/sgRNA plasmid and a donor gene plasmid to cells in vivo using SMNPs according to an embodiment of the invention.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

Some aspects of the invention include supramolecular nanoparticles (SMNPs), having a plurality of binding components, each having a plurality of binding regions; a plurality of cores that are suitable to at least provide some mechanical structure to the plurality of self-assembled supramolecular nanoparticles (SMNPs), the plurality of cores including at least one core binding element adapted to bind to the binding regions to form a first inclusion complex; and a plurality of terminating components, each having a single terminating binding element that binds to remaining binding regions of one of said plurality of binding components by forming a second inclusion complex. SMNPs are described in in U.S. Pat. No. 9,845,237 and U.S. Patent Application No. 20160000918, each of which is herein incorporated in its entirety by reference. The plurality of binding components, plurality of cores, and the plurality of terminating components self-assemble when brought into contact to form the supramolecular magnetic nanoparticle (SMNP).

The plurality of binding components, plurality of cores, and the plurality of terminating components bind to each other by one or more intermolecular forces. Examples of intermolecular forces include hydrophobic interactions, biomolecular interactions, hydrogen bonding interactions, π-π interactions, electrostatic interactions, dipole-dipole interactions, or van der Waals forces. Examples of biomolecular interactions include DNA hybridization, a protein-small molecule interaction (e.g. protein-substrate interaction (e.g. a streptavidin-biotin interaction) or protein-inhibitor interaction), an antibody-antigen interaction or a protein-protein interaction. Examples of other interactions include inclusion complexes or inclusion compounds, e.g. adamantane-β-cyclodextrin complexes or diazobenzene-α-cyclodextrin complexes, Generally, the intermolecular forces binding the components of the SMNP structure are not covalent bonds.

An embodiment of the invention relates to a method employing the combined use of non-viral vectors with a more effective homology-independent targeted integration (HITI) strategy to facilitate CRISPR/Cas9-mediated knockin of a full-length therapeutic gene or fragment thereof as a more effective and general non-viral therapeutic solution for many genetic diseases.

Some embodiments of the invention relate to compositions and methods for delivering a nucleic acid encoding an endonuclease and a nucleic acid sequence encoding a donor protein to a cell. In such embodiments, the composition includes a first plurality of self-assembled supramolecular nanoparticles (SMNPs) and a second plurality of self-assembled supramolecular nanoparticles (SMNPs). In such an embodiment, the nucleic acid encoding an endonuclease is encapsulated within each of the first plurality of self-assembled SMNPs, and the nucleic acid sequence encoding the donor protein is encapsulated within each of the second plurality of self-assembled SMNPs.

Some embodiments of the invention relate to compositions and methods for delivering an endonuclease and a donor protein to a cell. In such embodiments, the composition includes a first plurality of self-assembled SMNPs and a second plurality of self-assembled SMNPs. In such an embodiment, the endonuclease is encapsulated within each of the first plurality of self-assembled SMNPs, and the donor protein is encapsulated within each of the second plurality of self-assembled SMNPs.

As used throughout, the terms “donor protein” or “therapeutic protein” are used interchangeably and refer to a full length protein or fragment thereof serving as a functional replacement for a mutated or otherwise defective version of the protein or related gene endogenous to a cell or subject suffering from a genetic disorder associated with the mutated or defective protein or related gene. In some embodiments, the donor protein or fragment thereof is delivered into a cell or subject so that the donor protein or fragment thereof serves as a functional replacement for a mutated or otherwise defective version of the protein or related gene endogenous to the cell or subject.

In some embodiments, the donor protein or fragment thereof is delivered to a cell or subject in the form of a nucleic acid sequence encoding the donor protein or fragment thereof. In some embodiments, the nucleic acid sequence encoding the donor protein or fragment thereof is configured for insertion into the genomic DNA (gDNA) of a cell by homologous or non-homologous recombination. In some such embodiments, recombination of the nucleic acid sequence encoding the donor protein or fragment thereof into the gDNA of the host cell enables translation of donor protein or fragment thereof via the use of the cell's machinery.

In some embodiments, the nucleic acid sequence encoding the donor protein or fragment thereof forms part of a circular, double-stranded DNA molecule (e.g. a plasmid) and is encapsulated in a supra-molecular nanoparticle configured for delivery of the nucleic acid sequence encoding the donor protein or fragment and circular, double-stranded DNA molecule into a cell. Methods of preparing a plasmid including a nucleic acid sequence encoding a donor protein or fragment thereof are known in the art.

In some embodiments, a plasmid including a nucleic acid sequence encoding a donor protein or fragment thereof is encapsulated in a self-assembled SMNPs configured for delivery of the plasmid into a cell. In some such embodiments, the nucleic acid sequence encoding the donor protein or fragment thereof is configured for insertion into the gDNA of a cell by homologous or non-homologous recombination. In some such embodiments, the nucleic acid sequence encoding the donor protein or fragment thereof is configured for insertion into the gDNA of a cell specifically by non-homologous recombination.

In some embodiments, a nucleic acid sequence encoding a donor protein or fragment thereof is configured for insertion into the gDNA of a cell by a CRISPR/Cas9-mediated system and by non-homologous recombination. In such an embodiment, the CRISPR/Cas9-mediated gene editing system is composed of two functional components, i.e., Cas9 endonuclease and an engineered short, single-guide RNA (sgRNA). Based on a base-pairing mechanism, Cas9.sgRNA complex recognizes and cuts a targeted site, precisely inducing a double-strand break (DSB). In some embodiments, endogenous DNA repair then occurs via a non-homologous end joining (NHEJ) pathway. The nucleic acid sequence encoding the donor protein or fragment thereof is then integrated into DSB.

In some embodiments, a nucleic acid sequence encoding the donor protein or fragment thereof is configured for insertion into the genomic DNA of a cell by a CRISPR/Cas9-mediated system and by a homology-independent targeted integration (HITI) strategy, which was previously developed and based on the NHEJ pathway to enable robust knockin of non-dividing cells in vivo. In such embodiments, the HITI strategy introduces two pre-determined CRISPR/Cas9 target sites into the nucleic acid sequence. After cutting the targeted sites present in both the gDNA and in the nucleic acid sequence, the resulting three DSB sites undergoes endogenous DNA repair via the NHEJ pathway to achieve integration of the nucleic acid sequence into the gDNA.

Some embodiments of the invention relate to a method of treating a genetic disorder. In such embodiments, a nucleic acid sequence encoding a donor protein or fragment thereof is configured for insertion into the gDNA of a cell from a subject suffering from the genetic condition by a CRISPR/Cas9-mediated system. In some such embodiments, a nucleic acid sequence encoding Cas9 and a separate sgRNA are encapsulated in a first population of self-assembled SMNPs, and the nucleic acid sequence encoding a donor protein or fragment thereof is encapsulated in a second population of self-assembled SMNPs. In some such embodiments, both populations of self-assembled SMNPs are configured for uptake by cells from the subject. In some such embodiments, the cell(s) from the subject is contacted with both populations of self-assembled SMNPs such that at least one self-assembled SMNP from each population of self-assembled SMNPs is taken up by the cell(s). Then, the nucleic acid sequence encoding Cas9 is released from its SMNP and Cas9 is encoded. The Cas9 forms a complex with the sgRNA, and cuts a target site in the gDNA of the cell. The nucleic acid sequence encoding the donor protein or fragment thereof is also released from its SMNP, and is then integrated into the cell's gDNA at the target site via a NHEJ pathway. Once integrated, the donor protein or fragment thereof is then expressed and serves as a functional replacement for the mutated or otherwise defective version of the protein or related gene endogenous to the cell.

Some embodiments, relate to the method of treating a genetic disorder discussed above, where the genetic disorder is X-linked juvenile retinoschisis, Achromatopsia, Choroideremia, Leber congenital amaurosis, Retinitis pigmentosa, Usher syndrome type 1B, Neovascular AMD.

Some embodiments of the invention are related to compositions, systems, and/or methods for delivering CRISPR/Cas9-based genome editing system to a cell. In some embodiments, a self-assembled nano-particle is configured to encapsulate and deliver one or more functional Cas9 enzymes, Cpf1 enzymes and one or more guide RNAs to a cell for editing of a genomic DNA sequence (including, but not limited to a gene, and intron, and/or and exon). In some embodiments, a self-assembled nano-particle is configured to encapsulate and deliver a nucleic acid sequence encoding for a Cas9 enzyme or a Cpf1 enzyme. In some embodiments, a self-assembled nano-particle is configured to encapsulate and deliver a protein or peptide; non-limiting examples of such a protein or peptide include a recombinant protein or peptide, or a replacement protein or peptide. In some embodiments, a self-assembled nano-particle is configured to encapsulate and deliver a nucleotide sequence encoding a protein or a peptide.

Some embodiments of the invention are related to methods for genome editing in a cell. In some such embodiments, a target cell is contacted with a self-assembled nano-particle configured to encapsulate and deliver one or more functional Cas9 enzymes, Cpf1 enzymes and one or more guide RNAs to the cell for editing of a target genomic DNA sequence. In some embodiments, the self-assembled nano-particle is configured to encapsulate and deliver a nucleic acid sequence encoding for a Cas9 enzyme or a Cpf1 enzyme. In some embodiments, the target cell is contacted with two different self-assembled nano-particles: a first self-assembled nano-particle configured to encapsulate and deliver to the cell a functional Cas9 enzyme and a guide RNA, or a nucleic acid sequence encoding for a Cas9 enzyme; and a second self-assembled nano-particle configured to encapsulate and deliver a protein or peptide or a nucleic acid sequence encoding a protein or peptide.

Some embodiments of the invention include a composition having a plurality of self-assembled SMNPs, where the self-assembled SMNPs include a membrane penetration ligand. IN such embodiments, the membrane penetration ligand (e.g. TAT) is attached to the outer surface of the self-assembled SMNPs via in situ ligand dynamic exchange with adamantane-grafted polyethylene glycol (Ad-PEG) based on multivalent molecular recognition between b-cyclodextrin (CD) and adamantane (Ad) motifs. Non-limiting examples of concentrations or ratios of the Ad-PEG-TAT to Ad-PEG are from 1:100 to 50:100. Non-limiting examples of membrane penetrating ligands include TAT (GRKKRRQRRRPQ) (SEQ ID NO: 1), RGD (CRGDKGPDC) (SEQ ID NO:2), MPG (GLAFLGFLGAAGSTMGAWSQPKKKRKV) (SEQ ID NO:3), Pep-1 (KETW-WETWWTEWSQPKKRKV) (SEQ ID NO:4), GALA (WEAALAEALAEALAEHLAEALAEALEALAA) (SEQ ID NO:5), MAP17 (QLALQLALQALQAALQLA) (SEQ ID NO:6), and MAP12(LKTLTETLKELTKTLTEL) (SEQ ID NO:7). Additional example membrane penetrating ligands would be apparent to one of ordinary skill in the art.

An embodiment of the invention relates to a composition for delivering a nucleic acid encoding an endonuclease and a nucleic acid sequence encoding a donor protein to a cell including: a first plurality of self-assembled supramolecular nanoparticles (SMNPs), each of the first plurality of self-assembled supramolecular nanoparticles (SMNPs) including: a plurality of binding components, each having a plurality of binding regions; a plurality of cores that are suitable to at least provide some mechanical structure to the plurality of self-assembled supramolecular nanoparticles (SMNPs), the plurality of cores including at least one core binding element adapted to bind to the binding regions to form a first inclusion complex, wherein the plurality of binding components and the plurality of cores self-assemble when brought into contact to form the first plurality of self-assembled supramolecular nanoparticles (SMNPs); a plurality of terminating components, each having a single terminating binding element that binds to remaining binding regions of one of said plurality of binding components by forming a second inclusion complex, wherein the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle; the nucleic acid sequence encoding the endonuclease; and a nucleotide sequence including a recognition sequence specific to the endonuclease; and a second plurality of self-assembled supramolecular nanoparticles (SMNPs), each of the second plurality of self-assembled supramolecular nanoparticles (SMNPs) including: a plurality of binding components, each having a plurality of binding regions; a plurality of cores that are suitable to at least provide some mechanical structure to the plurality of self-assembled supramolecular nanoparticles (SMNPs), the plurality of cores including at least one core binding element adapted to bind to the binding regions to form a first inclusion complex, wherein the plurality of binding components and the plurality of cores self-assemble when brought into contact to form the second plurality of self-assembled supramolecular nanoparticles (SMNPs); a plurality of terminating components, each having a single terminating binding element that binds to remaining binding regions of one of said plurality of binding components by forming a second inclusion complex, wherein the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle; and the nucleic acid sequence encoding the donor protein. In such an embodiment, the nucleic acid sequence encoding the endonuclease and the nucleotide sequence including a recognition sequence specific to the endonuclease are encapsulated within each of the first plurality of self-assembled SMNPs, and the nucleic acid sequence encoding the donor protein is encapsulated within each of the second plurality of self-assembled SMNPs.

An embodiment of the invention relates to the composition above, where the endonuclease is a CRISPR associated protein 9 (Cas9), and the nucleotide sequence is a single guide RNA (sgRNA).

An embodiment of the invention relates to the composition above, where the plurality of cores and the plurality of binding components making up the first plurality of self-assembled SMNPs are present in a percent mass (w/w) ratio of between 0.5:1 and 3.0-1.

An embodiment of the invention relates to the composition above, where the plurality of cores and the plurality of binding components making up the second plurality of self-assembled SMNPs are present in a percent mass (w/w) ratio of between 0.5:1 and 3.0-1.

An embodiment of the invention relates to the composition above, where the plurality of terminating components of the first plurality of self-assembled SMNPs include a membrane penetration ligand.

An embodiment of the invention relates to the composition above, where the plurality of terminating components of the second plurality of self-assembled SMNPs include a membrane penetration ligand.

An embodiment of the invention relates to the composition above, where each of the first and second plurality of self-assembled supramolecular nanoparticles (SMNPs) has a diameter of between 40 nanometers and 600 nanometers.

An embodiment of the invention relates to the composition above, where the plurality of binding components of the first and second plurality of self-assembled SMNPs includes polythylenimine, poly(L-lysine), or poly(β-amino ester).

An embodiment of the invention relates to the composition above, where the plurality of binding regions of the first and second plurality of self-assembled SMNPs includes beta-cyclodextrin, alpha-cyclodextrin, gamma-cyclodextrin, cucurbituril or calixarene.

An embodiment of the invention relates to the composition above, where the plurality of cores of the first and second plurality of self-assembled SMNPs includes polyamidoamine dendrimers, poly(prophylenimine) (PPI) dendrimer, triazine dendrimer, carbosilane dendrimer, poly(ether imine) (PETIM) dendrimer or phosphorus dendrimer.

An embodiment of the invention relates to the composition above, where the at least one core binding element of the first and second plurality of self-assembled SMNPs includes adamantane, azobenzene, ferrocene or anthracene.

An embodiment of the invention relates to the composition above, where the plurality of terminating components of the first and second plurality of self-assembled SMNPs includes polyethylene glycol (PEG) or poly(propylene glycol) (PGG).

An embodiment of the invention relates to the composition above, where the single terminating binding element of the first and second plurality of self-assembled SMNPs includes adamantane, azobenzene, ferrocene or anthracene.

An embodiment of the invention relates to a method for delivering a nucleic acid encoding an endonuclease and a nucleic acid sequence encoding a donor protein to a cell including: providing a first plurality of self-assembled supramolecular nanoparticles (SMNPs); providing a second plurality of self-assembled SMNPs; and contacting the cell with at least one of the first plurality of self-assembled SMNPs and with at least one of the second plurality of self-assembled SMNPs, such that the at least one of the first plurality of self-assembled SMNPs and the at least one of the second plurality of self-assembled SMNPs are each taken up by the cell. In such an embodiment, each of the first plurality of self-assembled supramolecular nanoparticles SMNPs includes: a plurality of binding components, each having a plurality of binding regions; a plurality of cores that are suitable to at least provide some mechanical structure to the plurality of self-assembled SMNPs, the plurality of cores including at least one core binding element adapted to bind to the binding regions to form a first inclusion complex, wherein the plurality of binding components and the plurality of cores self-assemble when brought into contact to form the first plurality of self-assembled SMNPs; a plurality of terminating components, each having a single terminating binding element that binds to remaining binding regions of one of said plurality of binding components by forming a second inclusion complex, wherein the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle; the nucleic acid sequence encoding the endonuclease; and a nucleotide sequence including a recognition sequence specific to the endonuclease. In such an embodiment, each of the second plurality of self-assembled SMNPs includes: a plurality of binding components, each having a plurality of binding regions; a plurality of cores that are suitable to at least provide some mechanical structure to the plurality of self-assembled SMNPs, the plurality of cores including at least one core binding element adapted to bind to the binding regions to form a first inclusion complex, wherein the plurality of binding components and the plurality of cores self-assemble when brought into contact to form the second plurality of self-assembled SMNPs; a plurality of terminating components, each having a single terminating binding element that binds to remaining binding regions of one of said plurality of binding components by forming a second inclusion complex, wherein the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle; and the nucleic acid sequence encoding the donor protein. In such an embodiment, the nucleic acid sequence encoding the endonuclease and the nucleotide sequence including a recognition sequence specific to the endonuclease are encapsulated within each of the first plurality of self-assembled SMNPs, and the nucleic acid sequence encoding the donor protein is encapsulated within each of the second plurality of self-assembled SMNPs.

An embodiment of the invention relates to the method above, where the endonuclease is a CRISPR associated protein 9 (Cas9), and the nucleotide sequence is a single guide RNA (sgRNA).

An embodiment of the invention relates to the method above, where the plurality of cores and the plurality of binding components making up the first plurality of self-assembled SMNPs are present in a percent mass (w/w) ratio of between 0.5:1 and 3.0-1.

An embodiment of the invention relates to the method above, where the plurality of cores and the plurality of binding components making up the second plurality of self-assembled SMNPs are present in a percent mass (w/w) ratio of between 0.5:1 and 3.0-1.

An embodiment of the invention relates to the method above, where the plurality of terminating components of the first plurality of self-assembled SMNPs include a membrane penetration ligand.

An embodiment of the invention relates to the method above, where the plurality of terminating components of the second plurality of self-assembled SMNPs include a membrane penetration ligand.

An embodiment of the invention relates to the method above, where each of the first and second plurality of self-assembled supramolecular nanoparticles (SMNPs) has a diameter of between 40 nanometers and 600 nanometers.

An embodiment of the invention relates to the method above, where the plurality of binding components of the first and second plurality of self-assembled SMNPs includes polythylenimine, poly(L-Iysine), or poly(β-amino ester).

An embodiment of the invention relates to the method above, where the plurality of binding regions of the first and second plurality of self-assembled SMNPs includes beta-cyclodextrin, alpha-cyclodextrin, gamma-cyclodextrin, cucurbituril or calixarene.

An embodiment of the invention relates to the method above, where the plurality of cores of the first and second plurality of self-assembled SMNPs includes polyamidoamine dendrimers, poly(prophylenimine) (PPI) dendrimer, triazine dendrimer, carbosilane dendrimer, poly(ether imine) (PETIM) dendrimer or phosphorus dendrimer.

An embodiment of the invention relates to the method above, where the at least one core binding element of the first and second plurality of self-assembled SMNPs includes adamantane, azobenzene, ferrocene or anthracene.

An embodiment of the invention relates to the method above, where the plurality of terminating components of the first and second plurality of self-assembled SMNPs includes polyethylene glycol (PEG) or poly(propylene glycol) (PGG).

An embodiment of the invention relates to the method above, where the single terminating binding element of the first and second plurality of self-assembled SMNPs includes adamantane, azobenzene, ferrocene or anthracene.

### EXAMPLES

### Example 1

Homology-independent targeted integration (HITI) strategy is developed to enable effective CRISPR/Cas9-mediated knockin of a therapeutic gene in non-dividing cells in vivo, promising a general therapeutic solution for treating genetic diseases like X-linked juvenile retinoschisis, XLRS. Herein, supramolecular nanoparticle (SMNP) vectors are used for co-delivery of two DNA plasmids—CRISPR-Cas9 genome-editing system and a therapeutic gene, RS1—enabling CRISPR/Cas9 knockin of RS1 gene along the HITI strategy. Through small-scale combinatorial screenings, two SMNP vectors, i.e., Cas9/sgRNA-plasmid⊂SMNPs and Donor-RS1/GFP-plasmid⊂SMNPs with optimal delivery performances are identified. The identified SMNP vectors are then employed to carry out CRISPR/Cas9 knockin of RS1/GFP gene into the mouse Rosa26 safe-harbor site in vitro and in vivo. The in vivo study is performed by intravitreally injecting the two SMNP vectors into the mouse eyes, followed by repeated ocular imaging by a fundus camera and optical coherence tomography, as well as pathological and molecular analyses of the harvested retina tissues. The results indicate that i) mice ocular organs retained their anatomical integrity, ii) the precise integration of a single-copy 3.0-kb RS1/GFP gene into the Rosa26 site in the retinas, and iii) the expression of the integrated RS1/GFP gene in the retinas, thus demonstrating CRISPR/Cas9 knockin of RS1/GFP gene in mice retina.

Previously, a convenient and flexible self-assembled synthetic strategy for producing supramolecular nanoparticle^{[18]} (SMNP) vectors by mixing three molecular building blocks, i.e., β-cyclodextrin (CD)-grafted branched polyethyleneimine (CD-PEI), adamantane (Ad)-grafted polyamidoamine dendrimer (Ad-PAMAM), and Ad-grafted poly(ethylene glycol) (Ad-PEG) was demonstrated; methods for assembling such particles are described in U.S. Pat. No. 9,845,237 and U.S. Patent Application No. 20160000918, which are each herein incorporated in their entirety by reference. The multivalent Ad/CD molecular recognition allows modular control over the sizes, surface chemistry, and payloads of SMNP vectors, promising a diversity of imaging^{[18-19]} and therapeutic applications.^{[20]} It was demonstrated that this self-assembly strategy can be utilized for combinatorial formulation and screening of SMNPs to optimize formulations with significantly improved delivery performance.^{[21]}

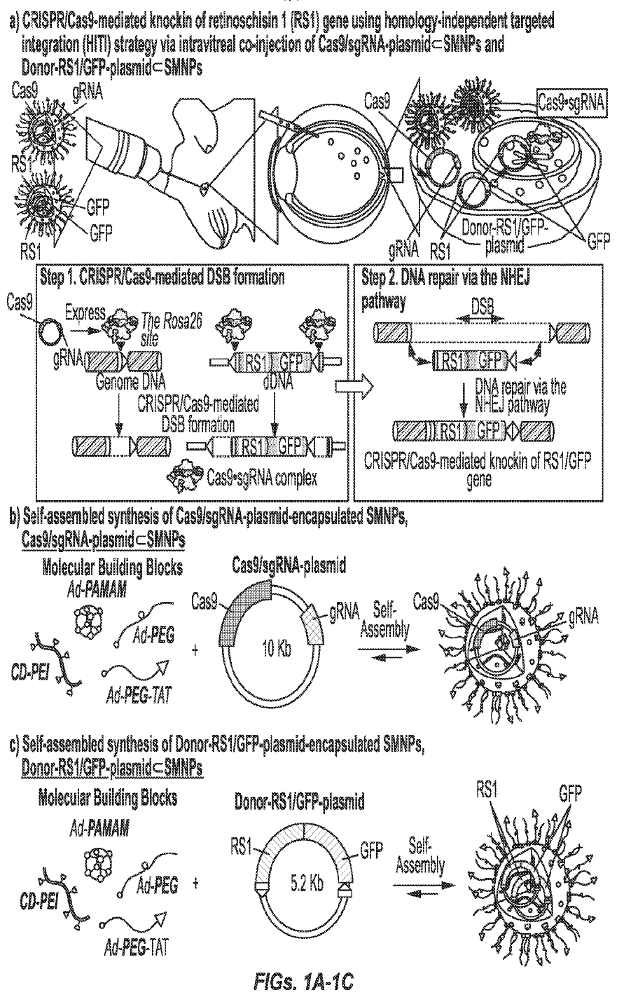

Herein, the combined use of SMNP vectors with the HITI strategy for in vivo CRISPR/Cas9-mediated knockin of RS1 gene (FIG. 1A) as a potential non-viral therapeutic solution for XLRS is described. By conducting small-scale combinatorial formulations and screenings,^{[21]} two SMNP vectors, i.e., Cas9/sgRNA-plasmid⊂SMNPs and Donor-RS1/GFP-plasmid⊂SMNPs with optimal delivery performance were identified. By performing intravitreal injection of the two SMNP vectors in a mouse model, Cas9/sgRNA-plasmid (10 kb) and Donor-RS1/GFP-plasmid (5.2 kb) can be introduced into the retina, initiating CRISPR/Cas9-mediated knockin of RS1/GFP gene in two consecutive steps. In Step 1, Cas9.sgRNA specifically recognizes and cuts a sgRNA-targeted sequence in a mouse Rosa26 safe-harbor site^{[23]} and two flanked sites adjacent to RS1/GFP genes (within the Donor-RS1/GFP-plasmid), resulting in the formation of three DSBs. In Step 2, DNA repair via the NHEJ pathway leads to site-specific integration of RS1/GFP genes. To prepare for the in vitro study, the B16 mouse melanoma cell line (no RS1 gene expression) was employed as a model system for optimization. The self-assembly strategy^{[18]} enables precise control over two synthetic variables,—i) Ad-PAMAM/CD-PEI ratios, and ii) the coverage of a membrane penetration ligand, TAT.^{[20c]} Through small-scale combinatorial screenings, optimal performances were identified for the formulations of Cas9/sgRNA-plasmid⊂SMNPs and Donor-RS1/GFP-plasmid⊂SMNPs according to their performances for inducing CRISPR/Cas9-mediated insertion and deletion (Indel) events and GFP transfection, respectively. The two optimized SMNP vectors were then employed to achieve CRISPR/Cas9-mediated knockin of RS1/GFP gene in growth-synchronized B16 cells. The resulting RS1/GFP-knockin B16 cells were further characterized by fluorescence microscopy, polymerase chain reaction (PCR) assay, Sanger sequencing, and quantitative PCR assay to confirm the successful integration of 3.0-kb RS1/GFP gene. Finally, the feasibility of performing CRISPR/Cas9-mediated knockin of RS1/GFP gene in mouse retina by intravitreally co-injecting the two SMNP into mice eyes is also described. Ocular imaging with a fundus camera and optical coherence tomography (OCT), as well as pathological and molecular analyses of the harvested retina tissues, were employed to test the success and efficiency CRISPR/Cas9-mediated knockin of RS1/GFP gene in mouse retinas.

FIGS. 1A-1C are illustrations showing example embodiments according to the invention. FIG. 1A is a schematic illustration showing that two supramolecular nanoparticle (SMNP) vectors were developed for co-delivery of Cas9/sgRNA-plasmid and Donor-RS1/GFP-plasmid, enabling CRISPR/Cas9-mediated knockin of RS1 gene in mouse retinas. After Cas9.sgRNA formation in vivo, CRISPR/Cas9-mediated knockin of RS1 gene is carried out in two consecutive steps following the homology-independent targeted integration (HITI) strategy. FIG. 1B is an illustration showing a self-assembled synthetic strategy adopted for preparation of Cas9/sgRNA-plasmid⊂SMNPs through stoichiometric mixing of 10-kb Cas9/sgRNA-plasmid and four SMNP molecular building blocks, i.e., CD-PEI, Ad-PAMAM, Ad-PEG, and Ad-PEG-TAT. FIG. 1C is an illustration showing a self-assembly strategy adopted for preparation of Donor-RS1/GFP-plasmid⊂SMNPs.

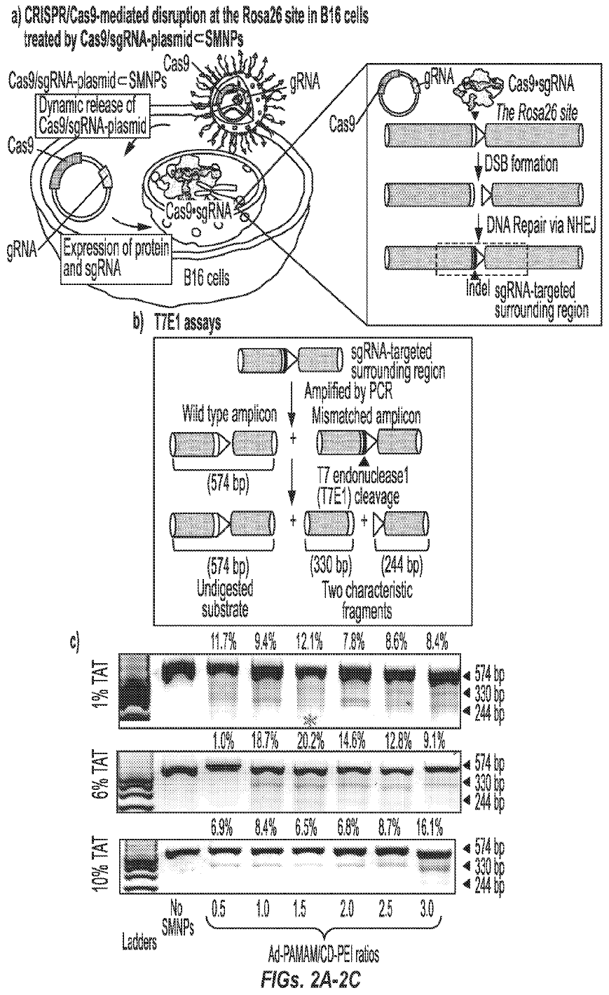

Based on the self-assembly strategy, a small-scale combinatorial screening approach was first adopted^{[21]} in search of a Cas9/sgRNA-plasmid⊂SMNPs formulation that optimized performance for CRISPR/Cas9-mediated disruption at the Rosa26 site (FIG. 2A). After cell uptake of Cas9/sgRNA-plasmid⊂SMNPs, Cas9.sgRNA was produced in cytoplasm, precisely introducing DSB at the Rosa26 site. The subsequent DNA repair via the NHEJ pathway led to the Indel events. For optimization, 18 formulations of Cas9/sgRNA-plasmid⊂SMNPs were prepared by systemically varying i) Ad-PAMAM/CD-PEI weight ratios=0.5 to 3.0, and ii) TAT ligand coverage=1 to 10%, while keeping the concentrations of Cas9/sgRNA-plasmid, Ad-PEG, and CD-PEI at 0.01, 0.23, and 0.1 μg/μL, respectively. Each formulation of Cas9/sgRNA-plasmid⊂SMNPs (containing 1.0 μg of Cas9/sgRNA-plasmid) was added to a well (in a 12-well plate), where 1×10⁵ B16 cells were starved in serum-free Dulbecco's modified Eagle's medium (DMEM) overnight to synchronize the cell cycles to GO/GI phases.^{[24]} Forty-eight h after SMNPs treatment, the B16 cells were subjected for genomic DNA extraction, and the sgRNA-targeted surrounding region was amplified by PCR. T7 endonuclease I (T7E1) assay^{[25]} (FIG. 2B) was performed to quantify the frequencies of the Indel events. T7 endonuclease specifically recognizes and cleaves mismatched DNA amplicons associated with the Indel events. Along with the wild-type (WT) amplicon (574 bp), two characteristic fragments (330 bp and 244 bp) were detected and quantified by electrophoretogram (FIG. 2C), reflecting the CRISPR/Cas9-mediated disruption performances of the 18 formulations. The optimal performance (20.2%) was identified for a Cas9/sgRNA-plasmid⊂SMNP formulation, of which Ad-PAMAM/CD-PEI is 1.5, and TAT coverage is 6%.

FIGS. 2A-2C are illustrations and eletrophoretograms showing optimization of self-assembling supramolecular nanoparticles (SMNPs) for delivering Cas9 according to an embodiment of the invention. FIG. 2A is a schematic illustration of CRISPR/Cas9-mediated disruption at the Rosa26 site in B16 cells treated by Cas9/sgRNA-plasmid⊂SMNPs. After cell uptake of SMNPs, Cas9.sgRNA was produced to introduce DSB precisely at the Rosa26 site. Subsequent DNA repair via the NHEJ pathway led to insertion and deletion (Indel) events. FIG. 2B is an illustration showing T7 endonuclease I (T7E1) assay employed to quantify the frequencies of the Indel events, reflecting the CRISPR/Cas9-mediated disruption performances. FIG. 2C is a series of electrophoretograms were used to quantify the two characteristic fragments (330 bp and 244 bp) associated with the Indel events along with the wild-type (WT) amplicon (574 bp). An optimal formulation of Cas9/sgRNA-plasmid⊂SMNPs was identified (*).

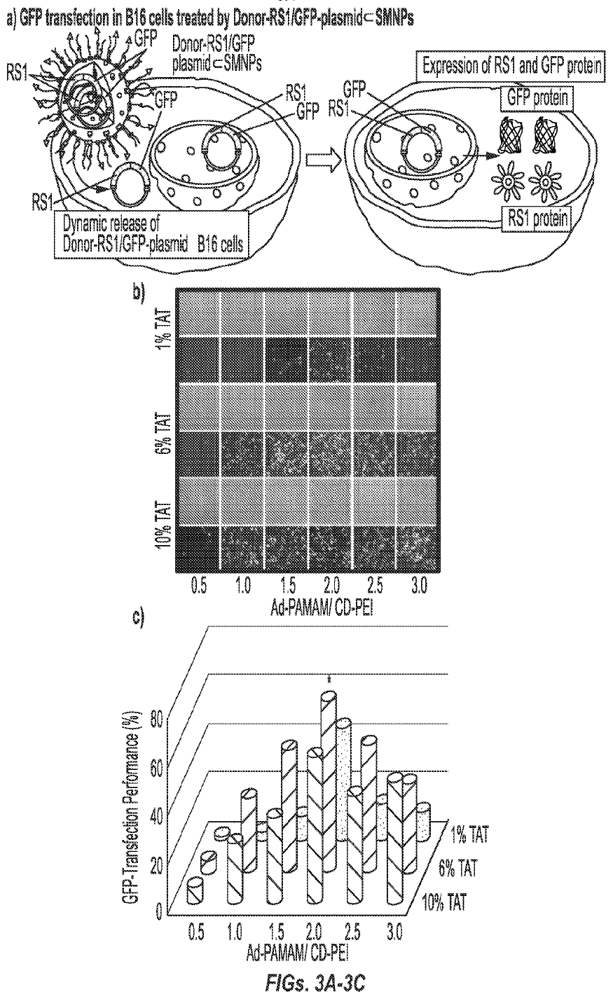

Based on a similar combinatorial screening approach,^{[21]} a Donor-RS1/GFP-plasmid⊂SMNPs formulation that optimizes GFP-transfection performance was evaluated (FIG. 3A). Eighteen formulations of Donor-RS1/GFP-plasmid⊂SMNPs were prepared by systemically varying i) Ad-PAMAM/CD-PEI weight ratios=0.5 to 3.0, ii) TAT ligand coverage=1 to 10%, while keeping the concentrations of Donor-RS1/GFP-plasmid, Ad-PEG, and CD-PEI at 0.01, 0.23, and 0.1 μg/μL, respectively. After settling growth-synchronized B16 cells in culture plates, each formulation of the SMNPs (containing 1.0 μg of Donor-RS1/GFP-plasmid) was added to the cells. Forty-eight h post SMNP treatment, fluorescence microscopy was used to quantify the GFP expression levels for individual formulations (FIG. 3B). The quantitative analysis summarized in FIG. 3C, revealed that the optimal GFP-transfection performance (70%) was identified for a Donor-RS1/GFP-plasmid⊂SMNPs formulation, where Ad-PAMAM/CD-PEI is 2.0, and TAT coverage is 6%.

FIGS. 3A-3C are illustrations, fluorescent images and a data chart showing optimization of self-assembling supramolecular nanoparticles (SMNPs) for delivering a donor gene plasmid according to an embodiment of the invention. FIG. 3A is a schematic illustration of green fluorescent protein (GFP) transfection in B16 cells treated by Donor-RS1/GFP-plasmid⊂SMNPs. FIG. 3B is a panel of images showing eighteen formulations of Donor-RS1/GFP-plasmid⊂SMNPs prepared for the GFP-transfection study, followed by fluorescence microscopy analysis. FIG. 3C is a chart showing quantitative analysis of the fluorescent micrographs revealed an optimal formulation (*) for Donor-RS1/GFP-plasmid⊂SMNPs.

Next, the feasibility of co-encapsulating both Cas9/sgRNA-plasmid and Donor-RS1/GFP-plasmid into a single SMNP vector was explored. Based on the previous formulation conditions, a Cas9/sgRNA-plasmid+Donor-RS1/GFP-plasmid⊂SMNPs was prepared via stoichiometric mixing of the two DNA plasmids with the SMNP building blocks. The resulting Cas9/sgRNA-plasmid+Donor-RS1/GFP-plasmid⊂SMNPs were subjected to both CRISPR/Cas9-mediated disruption and GFP-transfection studies. The results suggest that such co-encapsulated SMNP vector exhibited significantly compromised performance in CRISPR/Cas9-mediated disruption (2.8%) and GFP transfection (10%) (Data not shown).

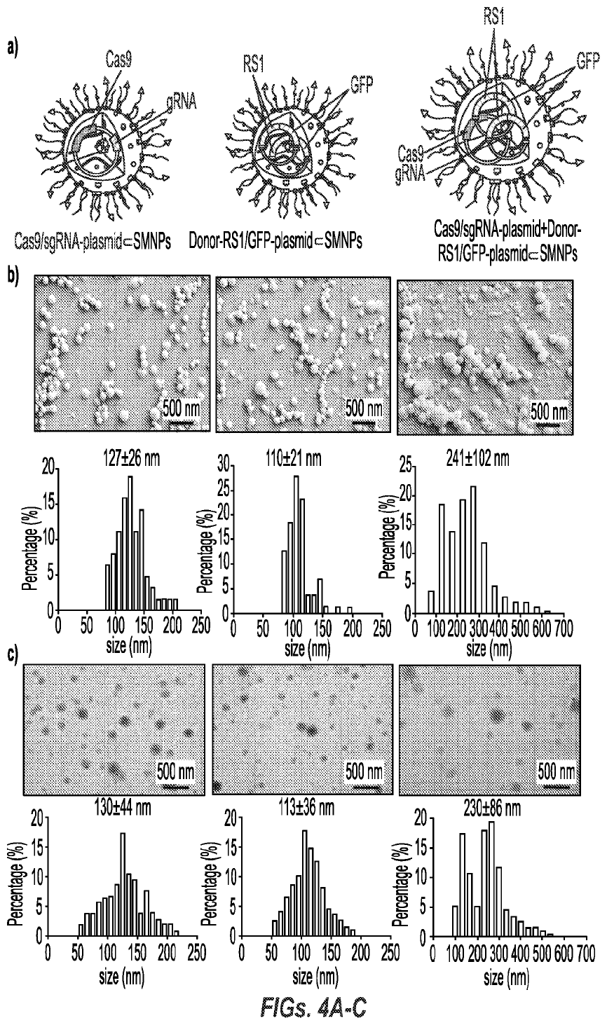

Scanning electron microscopy (SEM), transmission electron microscopy (TEM), and dynamic light scattering (DLS) analyses were used to characterize the optimized Cas9/sgRNA-plasmid⊂SMNPs, Donor-RS1/GFP-plasmid⊂SMNPs, and Cas9/sgRNA-plasmid+Donor-RS1/GFP-plasmid⊂SMNPs. The SEM and TEM micrographs and size distributions are shown in FIGS. 4A and 4B, and suggest that these two optimized formulations conferred homogeneous spherical morphologies with narrow size distributions to these two SMNP vectors. The co-encapsulated SMNPs have larger sizes and size distributions (FIGS. 4A and 4B), which might be responsible for their reduced performance. According to stoichiometric calculations, it was estimated that 1-2 copies of Cas9/sgRNA-plasmid and 2-3 copies of Donor-RS1/GFP-plasmid were encapsulated into each Cas9/sgRNA-plasmid⊂SMNP and Donor-RS1/GFP-plasmid⊂SMNP under the optimized formulation conditions, respectively.

FIGS. 4A-4C are illustrations, electron microscopy images, and data graphs showing optimization of SMNPs encapsulating a Cas9/sgRNA plasmid and a donor gene plasmid according to an embodiment of the invention. FIG. 4A is a schematic illustrations of optimal Cas9/sgRNA-plasmid⊂SMNPs, Donor-RS1/GFP-plasmid⊂SMNPs, and the co-encapsulated Cas9/sgRNA-plasmid+Donor-RS1/GFP-plasmid⊂SMNPs. FIG. 4B is a series of scanning electron microscopy (SEM) images, and FIG. 4C is a series of transmission electron microscopy (TEM) images summarizing the size distributions of these three SMNPs.

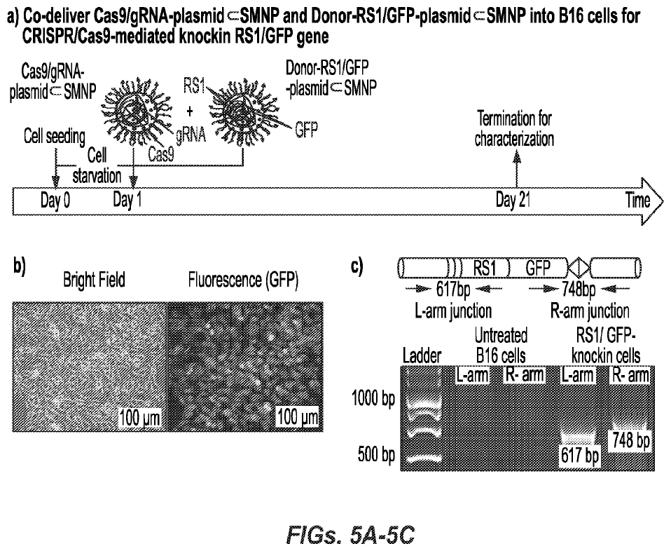

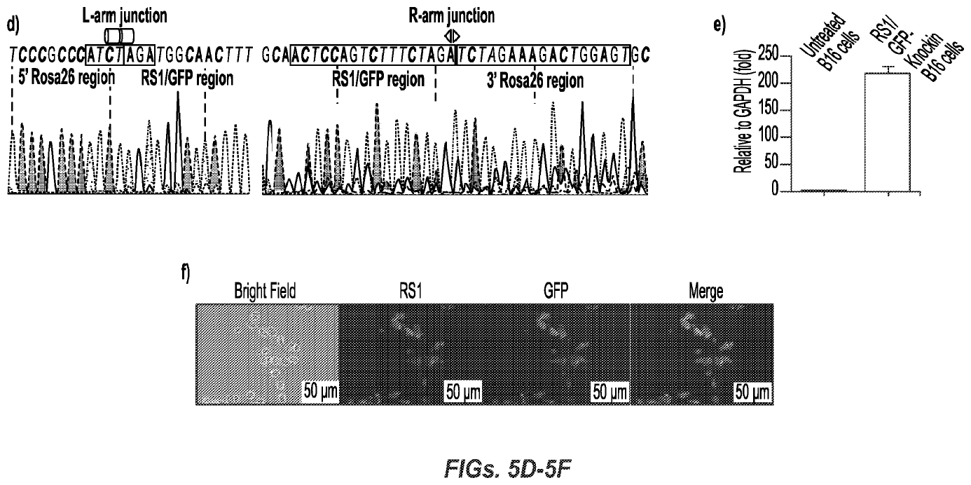

Using the two optimal SMNPs formulations, growth-synchronized B16 cells were treated by both Cas9/sgRNA-plasmid⊂SMNPs and Donor-RS1/GFP-plasmid⊂SMNPs (each containing 1.0 μg of plasmid) (FIG. 5A). Twenty-one days post treatment, the cells were subjected to flow cytometry analysis to determine the knockin efficiency (9.8%) and obtain purified RS1/GFP-knockin B16 cells. The purified RS1/GFP-knockin B16 cells were subjected to 20 rounds of culture expansion. Over the culture expansion, these cells exhibited stable and consistent GFP signals (FIG. 5B), supporting the integration of the RS1/GFP gene. To test the success of CRISPR-Cas9-mediated knockin of RS1/GFP gene into the Rosa26 site via the HITI pathway, the genomic DNA from RS1/GFP-knockin B16 cells was extracted, followed by PCR analysis and Sanger sequencing. After PCR amplification, the two characteristic DNA fragments, i.e., the L-arm junction (617 bp) and R-arm junction (748 bp)—signifying the integration of 3.0-kb RS1/GFP into the Rosa26 site—were detected by electrophoretogram (FIG. 5C). In parallel, Sanger sequencing of the genome-donor boundaries in the L-arm and R-arm junctions confirmed the successful integration of RS1/GFP gene via the HITI pathway (FIG. 5D). Together, these results indicate the successful CRISPR-Cas9-mediated knockin of RS1/GFP gene. Moreover, to examine whether the RS1/GFP-knockin B16 cells were capable of functionally expressing RS1 gene, quantitative RT-PCR analysis in RS1/GFP-knockin B16 cells was carried out using untreated B16 cells as controls. A significantly high level of RS1 expression in RS1/GFP-knockin B16 cells was observed (FIG. 5E). RS1/GFP-knockin B16 cells were further subjected to immunofluorescence (IF) staining to examine whether the integrated RS1 gene could functionally express RS1 protein. As shown in FIG. 5F, strong red fluorescence signals (marking IF-stained RS1 protein) were observed in RS1/GFP-knockin B16 cells. Collectively, the in vitro study suggested that the combined use of Cas9/sgRNA-plasmid⊂SMNPs and Donor-RS1/GFP-plasmid⊂SMNPs can successfully achieve CRISPR/Cas9-mediated knockin of a single-copy 3.0-kb RS1/GFP gene into the mouse Rosa26 site. These results represent the first demonstration of CRISPR/Cas9-mediated knockin of a long/intact gene using non-viral vector-based gene-delivery approach.

FIGS. 5A-5F are illustrations, fluorescent micrographs, and data graphs showing results of experiments assaying the delivery of a Cas9/sgRNA plasmid and a donor gene plasmid to cells in vitro using SMNPs according to an embodiment of the invention. FIG. 5A is a timeline depicting CRISPR/Cas9-mediated knockin of RS1/GFP gene in growth-synchronized B16 cells using both Cas9/sgRNA-plasmid⊂SMNPs and Donor-RS1/GFP-plasmid⊂SMNPs. FIG. 5B shows bright-field and fluorescence micrograph images of purified RS1/GFP-knockin B16 cells taken after 20 rounds of culture expansion. FIG. 5C is an illustration showing two characteristic DNA fragments, i.e., the L-arm junction (617 bp) and R-arm junction (748 bp)—signifying the integration of RS1/GFP into the Rosa26 site—were detected by an electrophoretogram. FIG. 5D shows Sanger sequencing carried out to test that the correct DNA sequences of the genome-donor boundaries in the L-arm and R-arm junctions. FIG. 5E shows RS1 gene expression levels observed by quantitative RT-PCR. FIG. 5F shows representative immunofluorescence images of RS1/GFP-knockin B16 cells.

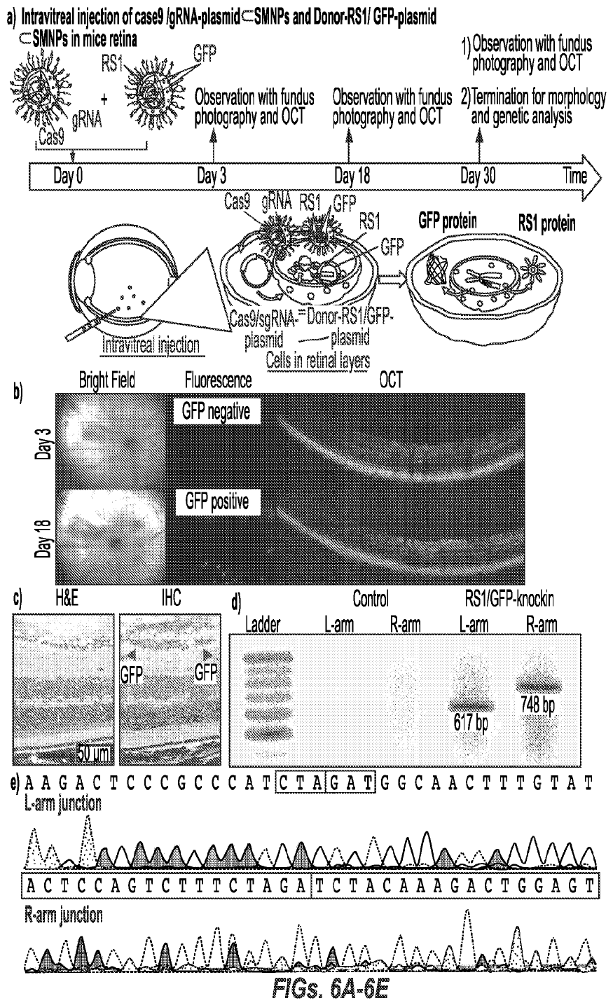

Following the successful demonstration of in vitro CRISPR/Cas9-mediated knockin, the feasibility of performing CRISPR/Cas9 knockin of RS1/GFP gene in vivo was assayed. A 5-uL phosphate-buffered saline (PBS) solution containing sterilized Cas9/sgRNA-plasmid⊂SMNPs and Donor-RS1/GFP-plasmid⊂SMNPs (each 50-ng plasmid) was injected intravitreally into the eye of a BALB/c-strain mouse (n=6) under brief isoflurane anesthesia. FIG. 6A shows the timeline of our in vivo study over a period of 30 days. At days 3, 10, and 30 post injection, a fundus camera and optical coherence tomography (OCT) were used to measure the knockin GFP signals on retinal surfaces (FIG. 6B left and middle panels) and to monitor the anatomical structures of the retinas (FIG. 6B right panel), respectively. Bright-field fundus and OCT imaging suggested that the mice retinas retained anatomical integrity over the course of the study. The GFP signals associated with CRISPR/Cas9-mediated knockin of RS1/GFP gene emerged at day 18 and persisted until day 30. At day 30, the mice were euthanized by cervical dislocation under deep anesthesia, and the treated eyes were excised for pathological and molecular analyses. After dissecting retinas from the treated eyes, the GFP-positive areas were subjected to standard pathology H&E staining and IHC staining for GFP (FIG. 6C). Two pathologists reviewed all the slides independently, concluding that: 1) no histological abnormalities were observed, and 2) GFP positivity was identified in the ganglion cell layers of the retinas. In parallel, genomic DNA was extracted from the GFP-positive retina tissues to confirm that the RS1/GFP gene was correctly integrated into the Rosa26 site. After PCR amplification, the two characteristic fragments, i.e., the L-arm junction (617 bp) and R-arm junction (748 bp) were observed on an electrophoretogram. (FIG. 6D). Sanger sequencing of the genome-donor boundaries in the L-arm and R-arm junctions indicated the successful integration of RS1/GFP gene. (FIG. 6E). Collectively, these in vivo experimental data support that the SMNP vectors can be utilized to deliver CRISPR/Cas9 gene editing system and dDNA using the HITI strategy, enabling CRISPR/Cas9-mediated knockin of intact RS1 gene in retina as a potential non-viral therapeutic solution for treating XLJR.

FIGS. 6A-6E are illustrations, fluorescent micrographs, and data graphs showing results of experiments assaying the delivery of a Cas9/sgRNA plasmid and a donor gene plasmid to cells in vivo using SMNPs according to an embodiment of the invention. FIG. 6A is a timeline and graphic illustration depicting CRISPR/Cas9-mediated knockin of the RS1/GFP gene in mouse retina via intravitreal injection of both Cas9/sgRNA-plasmid⊂SMNPs and Donor-RS1/GFP-plasmid⊂SMNPs. FIG. 6B is images from fundus camera and optical coherence tomography (OCT) employed to detect the GFP signals on retinal surfaces and monitor the anatomical structures of the retinas, respectively. FIG. 6C is H&E staining and IHC staining for GFP of the GFP-positive retina tissues. FIG. 6D shows two characteristic DNA fragments, i.e., the L-arm junction (617 bp) and R-arm junction (748 bp) on an electrophoretogram and FIG. 6E shows Sanger sequencing of the genome-donor boundaries in the L-arm and R-arm junctions confirmed the successful integration of 3.0-kb RS1/GFP gene into the Rosa26 site in vivo.

In summary, described above is an in vivo CRISPR/Cas9-mediated knockin approach using two SMNP vectors, i.e., Cas9/sgRNA-plasmid⊂SMNPs and Donor-RS1/GFP-plasmid⊂SMNPs, which were identified by performing small-scale combinatorial screenings of the different SMNP formulations prepared by a self-assembly synthetic strategy. By intravitreally injecting the two SMNP vectors in BALB/c-strain mice, it was successfully demonstrated that CRISPR/Cas9-mediated knockin of 3.0-kb RS1/GFP gene into the Rosa26 site in mice retinas is possible. This proof-of-concept study highlights the potential of the combined use of the SMNP vectors with the HITI strategy to achieve in vivo CRISPR/Cas9-mediated knockin of a therapeutic gene. Since nearly 200 mutation sites in the RS1 gene have been identified and associated with XLRS, it is not feasible to develop CRISPR/Cas9-mediated editing according to individual patients' mutations. Performing CRISPR/Cas9-mediated knockin of RS1 gene in the retinas of XLJR patients via either intravitreal or subretinal injection of the SMNPs would offer a revolutionary curative therapeutic solution. The same strategy should apply for treating other genetic diseases in which specific mutations only function in local organs (e.g., cystic fibrosis).

Materials and Methods.

Chemical reagents and solvents were purchased from Sigma and were used as received without further purification unless otherwise noted. Cas9/sgRNA-plasmid was a gift from Ralf Kuehn (Addgene plasmid #64216; http://n2t.net/addgene:64216; RRID:Addgene_64216). Donor-RS1/GFP-plasmid was synthesized from GeneCopoeia. B16 cells (mouse melanoma cell line) were purchased from American Type Culture Collection (ATCC) and maintained in Dulbeco's modified Eagle's medium (DMEM, Gibco) with 10% Fetal bovine serum (FBS, Gibco) and 1% penicillin-streptomycin (Gibco). For Indel events analysis: GeneArt® Genomic Cleavage Detection Kit was purchased from ThermoFisher. DNA extraction kit was purchased from Qiagen (QIAamp® DNA Mini Kit). All experimental procedures and protocols involving animals were approved by the institutional animal care committee of Taipei Veterans General Hospital and complied with the Guide for the Care and Use of Laboratory Animals.

Measurement.

Dynamic light scattering (DLS) was measured on Zetasizer Nano instrument (Malvern Instruments Ltd., United Kingdom) equipped with a 10-mW helium-neon laser (λ=632.8 nm) and a thermoelectric temperature controller. Measurements were taken at a 90° scattering angle.

Cell imaging studies were performed on a Nikon TE2000S inverted fluorescent microscope with a cooled charge-coupled device (CCD) camera (QImaging, Retiga 4000R), X-Cite 120 Mercury lamp, automatic stage, and filters for five fluorescent channels (W1: 325-375 nm, W2: 465-495 nm, W3: 570-590 nm, W4: 590-650 nm, and W5: 650-900 nm). Fluorescence intensities were measured by a Fujifilm BAS-5000 microplate reader.

Scanning electron microscope (SEM) images were performed on a TS-5136MM (TESCAN, Czech) scanning electron microscope at an accelerating voltage of 20 kV. Samples dispersed at an appropriate concentration were cast onto a glass sheet at room temperature and sputter-coated with gold.

Transmission electron microscope (TEM) images were carried out on a Philips CM 120 electron microscope, operating at an acceleration voltage of 120 kV. TEM samples were prepared by drop-coating 2 μL of sample suspension solutions onto carbon-coated copper grids. Excess amounts of solution were removed by filter papers after 45 s. Subsequently, the samples were negatively stained with 2% uranyl acetate for 45 s before TEM studies.

Cell Culture.

The mouse melanoma B16 cells were cultured in a humidified atmosphere of 5% CO₂/air in DMEM medium, supplemented with 10% fetal bovine serum, 100 units/ml penicillin and 100 μg/ml streptomycin. Individual wells of a 12-well plate were inoculated with complete medium containing 100,000 of B16 cells per well. The plates were incubated at 37° C. in a humidified 5% incubator for 18 h prior to the experiments.

Plasmid DNA.

The Cas9/sgRNA-plasmid was a gift from Ralf Kuehn (Addgene plasmid #64216; http://n2t.net/addgene:64216; RRID:Addgene_64216). The Donor-RS1/GFP-plasmid was purchased from GeneCopoeia.

Synthesis of Cas9/sgRNA-Plasmid⊂SMNPs.

A self-assembly procedure was applied to prepare the Cas9/sgRNA-plasmid encapsulated supramolecular nanoparticles (Cas9/sgRNA-plasmid⊂SMNPs). 18 Formulations of Cas9/sgRNA-plasmid⊂SMNPs were prepared via systemically modulating i) the weight ratios (0.5 to 3.0) between Ad-PAMAM and CD-PEI, ii) the percentages (1% to 10%) of TAT ligand on SMNP surfaces, while keeping the concentrations of Cas9/sgRNA-plasmid, Ad-PEG, and CD-PEI at 0.01, 0.23, and 0.1 μg/μL, respectively. The optimal synthesis formulation is below: The optimal synthesis formulation is below: A total of 2.0 μL DMSO solution containing Ad-PAMAM (15 μg) was added into a 100 μL PBS mixture with Cas9/sgRNA-plasmid (1.0 μg), Ad-PEG (23 μg), CD-PEI (10 μg), and Ad-PEG-TAT (1.4 μg). The above resulting mixture was then stirred vigorously to achieve optimal Cas9/sgRNA-plasmid⊂SMNPs. The mixture was stored at 4° C. for 1 h, after that, DLS and SEM and TEM were used to character the sizes of Cas9/sgRNA-plasmid⊂SMNPs.

Delivery Cas9/sgRNA-plasmid⊂SMNPs to B16 Cells.

Prior to settling the cells onto 12 well plate, B16 cells were starved in serum-free DMEM overnight (18 h) to synchronize cells to GO/GI phases of cell cycle.² B16 cells (1×10⁵) were introduced into each well of a 12-well plate. The Cas9/sgRNA-plasmid⊂SMNPs (containing 1.0 μg of Cas9/sgRNA-plasmid) was added to the well. The cells were co-incubated with SMNPs for 48 h.

T7E1 Assay.

The T7E1 assay was performed by using GeneArt™ Genomic Cleavage Detection Kit (purchased from ThermFisher, A24372). In brief, genomic DNA of the transfected cells were extracted by Cell Lysis Buffer. PCR products were purified with AmpliTaq Gold® 360 Master Mix and were denatured and annealed by using S1000™ Thermal Cycler (Bio-Rad). Hybridized PCR products were digested with Detection Enzyme at 37° C. for 1 hour and subjected to 2% agarose gel electrophoresis.

The PCR primer sequences were:

Rosa26_T7E1_F:
(SEQ ID NO: 8)
TACTCCGAGGCGGATCACAA
Rosa26_T7E1_R:
(SEQ ID NO: 9)
GCAAGCACGTTTCCGACTTG

Synthesis of Donor-RS1/GFP-Plasmid⊂SMNPs.

The similar self-assembly procedure was applied to prepare the Donor-RS1/GFP-plasmid⊂SMNPs. 18 Formulations of Donor-RS1/GFP-plasmid⊂SMNPs was prepared via systemically modulating i) the weight ratios (0.5 to 3.0) between Ad-PAMAM and CD-PEI, ii) the percentages (1% to 10%) of TAT ligand on SMNP surfaces, while keeping the concentrations of Donor-RS1/GFP-plasmid, Ad-PEG, and CD-PEI at 0.01, 0.23, and 0.1 μg/μL, respectively. The optimal synthesis formulation is below: The optimal synthesis formulation is below: A total of 2.0 μL DMSO solution containing Ad-PAMAM (20 μg) was added into a 100 μL PBS mixture with Cas9/GFP-plasmid (1.0 μg), Ad-PEG (23 μg), CD-PEI (10 μg), and Ad-PEG-TAT (1.4 μg). The above resulting mixture was then stirred vigorously to achieve optimal Donor-RS1/GFP-plasmid⊂SMNPs. The mixture was stored at 4° C. for 30 min, after that, DLS, SEM and TEM were used to character the sizes of EGFP-Cas9.sgRNA⊂SMNPs.

Delivery Donor-RS1/GFP-Plasmid⊂SMNPs into B16 Cells.

Prior to settling the cells onto 12 well plate, B16 cells were starved in serum-free DMEM overnight (18 h) to synchronize cells to GO/GI phases of cell cycle. B16 cells (1×10⁵) were introduced into each well of a 12-well plate. The Donor-RS1/GFP-plasmid⊂SMNPs (containing 1.0 μg of Donor-RS1/GFP-plasmid) was added to the well. The cells were co-incubated with SMNPs for 48 h. Microscopy-based image cytometry was used to detect the cellular uptake performances of different formulations. After different treatments, the GFP signal was quantified with fluorescent microscope with a CCD camera (Nikon H550, Japan).

Synthesis of Cas9/sgRNA-Plasmid+Donor-RS1/GFP-Plasmid⊂SMNPs.

The similar self-assembly procedure was applied to prepare the Donor-RS1/GFP-plasmid⊂SMNPs. The synthesis formulation is below: A total of 4.0 μL DMSO solution containing Ad-PAMAM (35 μg) was added into a 200 μL PBS mixture with Cas9/sgRNA-plasmid (1.0 μg), Donor-RS1/GFP-plasmid (1.0 μg), Ad-PEG (46 μg), CD-PEI (20 μg), and Ad-PEG-TAT (2.8 μg). The above resulting mixture was then stirred vigorously to achieve Cas9/sgRNA-plasmid+Donor-RS1/GFP-plasmid⊂SMNPs. The mixture was stored at 4° C. for 30 min, after that, DLS, SEM and TEM were used to character the sizes of SMNPs.

Delivery Cas9/sgRNA-Plasmid+Donor-RS1/GFP-Plasmid⊂SMNPs into B16 Cells.

Prior to settling the cells onto 12 well plate, B16 cells were starved in serum-free DMEM overnight (18 h) to synchronize cells to GO/GI phases of cell cycle.² B16 cells (1×10⁵) were introduced into each well of a 12-well plate. The Cas9/sgRNA-plasmid+Donor-RS1/GFP-plasmid ⊂SMNPs (containing 1.0 μg of Cas9/sgRNA-plasmid and 1.0 μg of Donor-RS1/GFP-plasmid) were added to the well. The cells were co-incubated with SMNPs for 48 h. Microscopy-based image cytometry was used to detect the cellular uptake performances of different formulations. After different treatments, the GFP signal was quantified with fluorescent microscope with a CCD camera (Nikon H550, Japan). T7E1 assay was performed to quantify the frequencies of the Indel events.

Stoichiometric Calculations for the Number of Cas9/sgRNA-Plasmid and Donor-RS1/GFP-Plasmid Encapsulated into Each Cas9/sgRNA-Plasmid⊂SMNP and Donor-RS1/GFP-Plasmid⊂SMNP, Respectively.

1. The total number of Cas9/sgRNA-plasmid⊂SMNPs, n_{vector}:

$${n}_{vector} = \frac{{m}_{total vectors}}{{m}_{vector}} = \frac{{m}_{total vectors}}{\frac{4}{3} π {r}^{3} ρ} \qquad ( 1 )$$

where m_{total vectors} is the total mass of Cas9/sgRNA-plasmid⊂SMNPs (50.4 μg), r is the radius of SMNP vector (65 nm), p is the density of SMNP vector (1.1 g/cm³). By calculation, n_{vector}=4×10¹⁰.

The total number of Cas9/sgRNA-plasmid, n_{Cas9/sgRNA-plasmid}.

$${n}_{Cas 9 / sgRNA - plasmid} = \frac{{m}_{total Cas 9 / sgRNA - plasmid}}{{M}_{Cas 9 / sgRNA - plasmid}} {N}_{A} \qquad ( 2 )$$

where m_{total Cas9/sgRNA-plasmid} is the total mass of Cas9/sgRNA-plasmid (1.0 μg), M_{Cas9/sgRNA-plasmid} is the molecular weight of Cas9/sgRNA-plasmid (10 kb≈6,600 kDa), N_{A} is the Avogadro constant (6.02×10²³). By calculation, n_{Cas9/sgRNA-plasmid}=9.1×10¹⁰.

The number of Cas9/sgRNA-plasmid encapsulated into each Cas9/sgRNA-plasmid⊂SMNP, n_{Cas9/sgRNA-plasmid/vector}:

$${n}_{Cas 9 / sgRNA - plasmid / vector} = \frac{{n}_{Cas 9 / sgRNA - plasmid}}{{n}_{vector}} \approx 2 \qquad ( 3 )$$

2. The total number of Donor-RS1/GFP-plasmid⊂SMNPs, n_{vector}:

$${n}_{vector} = \frac{{m}_{total vectors}}{{m}_{vector}} = \frac{{m}_{total vectors}}{\frac{4}{3} π {r}^{3} ρ} \qquad ( 4 )$$

where m_{total vectors} is the total mass of Donor-RS1/GFP-plasmid⊂SMNPs (55.4 μg), r is the radius of SMNP vector (55 nm), ρ is the density of SMNP vector (1.1 g/cm³). By calculation, n_{vector}=7.1×10¹⁰.

The total number of Donor-RS1/GFP-plasmid, n_{Donor-RS1/GFP-plasmid}:

$${n}_{Donor - RS 1 / GFP - plasmid} = \frac{{m}_{totalDonor - RS 1 / GFP - plasmid}}{{M}_{Donor - RS 1 / GFP - plasmid}} {N}_{A} \qquad ( 5 )$$

where m_{total Donor-RS1/GFP-plasmid} is the total mass of Donor-RS1/GFP-plasmid (1.0 μg), M_{Donor-RS1/GFP-plasmid} is the molecular weight of Donor-RS1/GFP-plasmid (5.2 kb≈3,200 kDa), N_{A} is the Avogadro constant (6.02×10²³). By calculation, n_{Donor-RS1/GFP-plasmid}=1.8×10¹¹.

The number of Donor-RS1/GFP-plasmid encapsulated into each Donor-RS1/GFP-plasmid⊂SMNP, n_{Donor-RS1/GFP-plasmid/vector}:

$${n}_{Donor - RS 1 / GFP - plasmid / vector} = \frac{{n}_{Donor - RS 1 / GFP - plasmid}}{{n}_{vector}} \approx 3 \qquad ( 6 )$$

Co-Delivery Cas9/sgRNA-Plasmid⊂SMNPs and Donor-RS1/GFP-Plasmid⊂SMNPs to B16 Cells.

Using the two optimal SMNPs formulations, growth-synchronized B16 cells (1×10⁵) were co-delivered by both Cas9/sgRNA-plasmid⊂SMNPs and Donor-RS1/GFP-plasmid⊂SMNPs (each containing 1.0 μg of plasmid) for 48 h. 21 Days post treatment, the cells were subjected to flow cytometry analysis to determine the knockin efficiency and obtain purified RS1/GFP-knockin B16 cells.

DNA Extraction and PCR.

The RS1/GFP-knockin B16 cells were harvested and then washed with PBS. The genomic DNA was extracted with a commercial QIAamp® DNA Mini Kit (Qiagen, Germany), following manufacturer's instructions. Then, PCR was conducted to amplify integrated RS1/GFP gene with a S1000™ Thermal Cycler (Bio-Rad) under the following PCR conditions: 95° C. for 10 minutes followed by 40 cycles (95° C. for 15 s, 55° C. for 15 s and 72° C. for 30 s) and 72° C. for 5 minutes. The PCR products were checked on a 1.5% electrophoresis gel.

The PCR primer sequences were listed as follow:

L junction_F:
(SEQ ID NO: 10)
ATGCCAATGCTCTGTCTAGGG
L junction_R:
(SEQ ID NO: 11)
TTCTCTAGGCACCGGTTCAAT
R junction_F:
(SEQ ID NO: 12)
CATCATCTCCCGCTTCATCCG
R junction_R:
(SEQ ID NO: 13)
CAAGCACGTTTCCGACTTGA

Quantitative PCR.

After adding TRIzol (800 μL), the cells were homogenized, treated with chloroform (160 μL) and centrifuged for 15 min at 4° C. The aqueous phase of the sample was removed by pipet and 100% isopropanol (400 μL) was added. After being centrifuged for 10 min, the supernatant was removed from the tube, and the pellet was washed with 75% ethanol and centrifuged for 5 min. Afterwards, the supernatant was removed, and the pellet was dissolved in DNase- and RNase-free water. RNA (1 μg) was reverse transcribed using the SuperScript III First-Strand Synthesis kit. qPCR analysis was performed using PowerUp SYBR Green Master Mix (Applied Biosystems) with the primers. Values were normalized against the gene expression of the housekeeping gene Gapdh.

The qPCR primer sequences were listed as follow:

RS1_F_q:
(SEQ ID NO: 14)
GATTGCCAAGGAGGACCCAA
RS1_R_q:
(SEQ ID NO: 15)
GACCTCCCCTGACTCGAAAC
Gapdh_F_q:
(SEQ ID NO: 16)
TGTGAACGGATTTGGCCGTA
Gapdh_R_q:
(SEQ ID NO: 17)
ACTGTGCCGTTGAATTTGCC

Immunofluorescence Staining.

The living cells were fixed in 4% paraformaldehyde, permeabilized in 0.1% Triton X-100, and blocked in 5% normal bovine serum albumin (BSA) in PBS. The cells were incubated with RS1 (1:500; Abcam) and GFP (1:500; Cell Signaling Technology) antibody. After being washed three times with PBS, the cells were incubated with secondary antibodies conjugated with FITC (green) and Cy3 (red). DAPI (blue) was used as the nuclear stain. Labeled cells were imaged with a laser-scanning confocal microscope (Olympus). The total amount of retained immunofluorescent material was determined in the green (488 nm) and the red (546) channels.

Intravitreal Injection.

C57BL/6 male mice (6-10 weeks old) were purchased from National Laboratory Animal Center (Taipei, Taiwan). The mice were housed in a pathogen-free space and operated according to the National Research Council's Guide for the Care and Use of Laboratory.

Animals. All anesthesia and sacrifice procedures were reviewed and approved by the Animal Care and Use Committee of the Taipei Veterans General Hospital (TVGH). The mice were anesthetized with 250 mg/kg tribromoethanol (Sigma-Aldrich) by intraperitoneal injection, and placed under a dissecting microscope (SZX16, OLYMPUS, Japan) or spectral-domain OCT imaging system. Each mouse was intravitreally injected with 5 μl of optimal Cas9/sgRNA-plasmid⊂SMNPs and Donor-RS1/GFP-plasmid⊂SMNPs into both eyes. A Hamilton syringe was used to inject 5 μl of the vectors into the vitreous cavity of an eye through the sclera behind the limbus of mice. The OCT images of the mouse retinas were obtained using a continuous, high-speed and high-resolution retinal image acquisition system (axial resolution, 7 μm; acquisition speed, 76 frames/s, 1000×1024 pixels in the X-Z plane). A horizontal scan of 400 images was obtained through the fundus.

H&E Staining and IHC.

After 30 days of injection, the mice eyes were collected and fixed with 4% paraformaldehyde. The paraffin-embedded tissue was sectioned and stained with hematoxylin and eosin (H&E). The paraffin embedded sections were deparaffinized and rehydrated in Target Retrieval Solution (DaKo). Sections were blocked with 3% fetal bovine serum for 5 mins and incubated with the primary antibodies for 30 mins at room temperature. The primary antibodies used in this assay were anti-GFP (1:100; Cell Signaling).

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. Moreover, features described in connection with one embodiment of the invention may be used in conjunction with other embodiments, even if not explicitly stated above. It is therefore to be understood that, within the scope of the claims and their equivalents, the invention may be practiced otherwise than as specifically described.

### REFERENCES

[1] a) F. A. Ran, P. D. Hsu, J. Wright, V. Agarwala, D. A. Scott, F. Zhang, Nat. Protoc. 2013, 8, 2281; b) J. A. Doudna, E. Charpentier, Science 2014, 346, 1258096; c) F. Jiang, J. A. Doudna, Annu. Rev. Biophys. 2017, 46, 505.
[2] a) G. Schwank, B. K. Koo, V. Sasselli, J. F. Dekkers, I. Heo, T. Demircan, N. Sasaki, S. Boymans, E. Cuppen, C. K. van der Ent, E. E. Nieuwenhuis, J. M. Beekman, H. Clevers, Cell Stem Cell 2013, 13, 653; b) Y. Wu, D. Liang, Y. Wang, M. Bai, W. Tang, S. Bao, Z. Yan, D. Li, J. Li, Cell Stem Cell 2013, 13, 659.
[3] a) V. T. Chu, T. Weber, B. Wefers, W. Wurst, S. Sander, K. Rajewsky, R. Kühn, Nat. Biotechnol. 2015, 33, 543; b) K. Schumann, S. Lin, E. Boyer, D. R. Simeonov, M. Subramaniam, R. E. Gate, G. E. Haliburton, J. Y. Chun, J. A. Bluestone, J. A. Doudna, Proc. Natl. Acad. Sci. U.S.A. 2015, 112, 10437; c) D. Paquet, D. Kwart, A. Chen, A. Sproul, S. Jacob, S. Teo, K. M. Olsen, A. Gregg, S. Noggle, M. Tessier-Lavigne, Nature 2016, 533, 125; d) J. Eyquem, J. Mansilla-Soto, T. Giavridis, S. J. van der Stegen, M. Hamieh, K. M. Cunanan, A. Odak, M. Gonen, M. Sadelain, Nature 2017, 543, 113.
[4] A. Orthwein, S. M. Noordermeer, M. D. Wilson, S. Landry, R. I. Enchev, A. Sherker, M. Munro, J. Pinder, J. Salsman, G. Dellaire, B. Xia, M. Peter, D. Durocher, Nature 2015, 528, 422.
[5] a) K. Suzuki, Y. Tsunekawa, R. Hernandez-Benitez, J. Wu, J. Zhu, E. J. Kim, F. Hatanaka, M. Yamamoto, T. Araoka, Z. Li, M. Kurita, T. Hishida, M. Li, E. Aizawa, S. Guo, S. Chen, A. Goebl, R. D. Soligalla, J. Qu, T. Jiang, X. Fu, M. Jafari, C. R. Esteban, W. T. Berggren, J. Lajara, E. Nunez-Delicado, P. Guillen, J. M. Campistol, F. Matsuzaki, G. H. Liu, P. Magistretti, K. Zhang, E. M. Callaway, K. Zhang, J. C. Belmonte, Nature 2016, 540, 144; b) X. He, C. Tan, F. Wang, Y. Wang, R. Zhou, D. Cui, W. You, H. Zhao, J. Ren, B. Feng, Nucleic Acids Res. 2016, 44, e85.
[6] A. Tantri, T. R. Vrabec, A. Cu-Unjieng, A. Frost, W. H. Annesley, Jr., L. A. Donoso, Surv. Ophthalmol. 2004, 49, 214.
[7] G. A. Rodrigues, E. Shalaev, T. K. Karami, J. Cunningham, N. K. H. Slater, H. M. Rivers, Pharm. Res. 2018, 36, 29.
[8] C. Cukras, H. E. Wiley, B. G. Jeffrey, H. N. Sen, A. Turriff, Y. Zeng, C. Vijayasarathy, D. Marangoni, L. Ziccardi, S. Kjellstrom, T. K. Park, S. Hiriyanna, J. F. Wright, P. Colosi, Z. Wu, R. A. Bush, L. L. Wei, P. A. Sieving, Mol. Ther. 2018, 26, 2282.
[9] L. Swiech, M. Heidenreich, A. Banerjee, N. Habib, Y. Li, J. Trombetta, M. Sur, F. Zhang, Nat. Biotechnol. 2015, 33, 102.
[10] Z. Wu, H. Yang, P. Colosi, Mol. Ther. 2010, 18, 80.
[11] a) H.-X. Wang, M. Li, C. M. Lee, S. Chakraborty, H.-W. Kim, G. Bao, K. W. Leong, Chem. Rev. 2017, 117, 9874; b) L. Li, S. Hu, X. Chen, Biomaterials 2018, 171, 207.
[12] a) J. A. Zuris, D. B. Thompson, Y. Shu, J. P. Guilinger, J. L. Bessen, J. H. Hu, M. L. Maeder, J. K. Joung, Z.-Y. Chen, D. R. Liu, Nat. Biotechnol. 2015, 33, 73; b) M. Wang, J. A. Zuris, F. Meng, H. Rees, S. Sun, P. Deng, Y. Han, X. Gao, D. Pouli, Q. Wu, Proc. Natl. Acad. Sci. U.S.A. 2016, 113, 2868.
[13] a) L. Li, L. Song, X. Liu, X. Yang, X. Li, T. He, N. Wang, S. Yang, C. Yu, T. Yin, ACS Nano 2016, 11, 95; b) W. Sun, W. Ji, J. M. Hall, Q. Hu, C. Wang, C. L. Beisel, Z. Gu, Angew. Chem. Int. Ed. 2015, 54, 12029; c) Q. Liu, K. Zhao, C. Wang, Z. Zhang, C. Zheng, Y. Zhao, Y. Zheng, C. Liu, Y. An, L. Shi, Adv. Sci. 2019, 6.
[14] a) W. Zhou, H. Cui, L. Ying, X. F. Yu, Angew. Chem. Int. Ed. 2018, 130, 10425; b) P. Wang, L. Zhang, W. Zheng, L. Cong, Z. Guo, Y. Xie, L. Wang, R. Tang, Q. Feng, Y. Hamada, Angew. Chem. Int. Ed. 2018, 57, 1491; c) B. Lee, K. Lee, S. Panda, R. Gonzales-Rojas, A. Chong, V. Bugay, H. M. Park, R. Brenner, N. Murthy, H. Y. Lee, Nat. Biomed. Eng. 2018, 2, 497; d) K. Lee, M. Conboy, H. M. Park, F. Jiang, H. J. Kim, M. A. Dewitt, V. A. Mackley, K. Chang, A. Rao, C. Skinner, Nat. Biomed. Eng. 2017, 1, 889.
[15] a) X. Gao, Y. Tao, V. Lamas, M. Huang, W.-H. Yeh, B. Pan, Y.-J. Hu, J. H. Hu, D. B. Thompson, Y. Shu, Nature 2018, 553, 217; b) S. K. Alsaiari, S. Patil, M. Alyami, K. O. Alamoudi, F. A. Aleisa, J. S. Merzaban, M. Li, N. M. Khashab, J Am. Chem. Soc. 2017, 140, 143.
[16] P. Mora-Raimundo, D. Lozano, M. Manzano, M. Vallet-Regi, ACS Nano 2019.
[17] a) H.-X. Wang, Z. Song, Y.-H. Lao, X. Xu, J. Gong, D. Cheng, S. Chakraborty, J. S. Park, M. Li, D. Huang, Proc. Natl. Acad. Sci. U.S.A. 2018, 115, 4903; b) H. Yin, C.-Q. Song, J. R. Dorkin, L. J. Zhu, Y. Li, Q. Wu, A. Park, J. Yang, S. Suresh, A. Bizhanova, Nat. Biotechnol. 2016, 34, 328; c) J. Carlson-Stevermer, A. A. Abdeen, L. Kohlenberg, M. Goedland, K. Molugu, M. Lou, K. Saha, Nat. Commun. 2017, 8, 1711.
[18] H. Wang, S. Wang, H. Su, K. J. Chen, A. L. Armijo, W. Y. Lin, Y. Wang, J. Sun, K. i. Kamei, J. Czernin, Angew. Chem. Int. Ed. 2009, 48, 4344.
[19] K.-J. Chen, S. M. Wolahan, H. Wang, C.-H. Hsu, H.-W. Chang, A. Durazo, L.-P. Hwang, M. A. Garcia, Z. K. Jiang, L. Wu, Biomaterials 2011, 32, 2160.
[20] a) J.-s. Choi, Y. Zhu, H. Li, P. Peyda, T. T. Nguyen, M. Y. Shen, Y. M. Yang, J. Zhu, M. Liu, M. M. Lee, ACS Nano 2016, 11, 153; b) J. H. Lee, K. J. Chen, S. H. Noh, M. A. Garcia, H. Wang, W. Y. Lin, H. Jeong, B. J. Kong, D. B. Stout, J. Cheon, Angew. Chem. Int. Ed. 2013, 52, 4384; c) S. Wang, K. J. Chen, T. H. Wu, H. Wang, W. Y. Lin, M. Ohashi, P. Y. Chiou, H. R. Tseng, Angew. Chem. Int. Ed. 2010, 49, 3777.
[21] a) H. Wang, K. Liu, K. J. Chen, Y. Lu, S. Wang, W. Y. Lin, F. Guo, K. Kamei, Y. C. Chen, M. Ohashi, M. Wang, M. A. Garcia, X. Z. Zhao, C. K. Shen, H. R. Tseng, ACS Nano 2010, 4, 6235; b) H. Wang, K. J. Chen, S. Wang, M. Ohashi, K. Kamei, J. Sun, J. H. Ha, K. Liu, H. R. Tseng, Chem. Commun. 2010, 46, 1851.
[22] Y. Liu, J. Du, J. s. Choi, K. J. Chen, S. Hou, M. Yan, W. Y. Lin, K. S. Chen, T. Ro, G. S. Lipshutz, Angew. Chem. Int. Ed. 2016, 55, 169.
[23] E. P. Papapetrou, A. Schambach, Mol. Ther. 2016, 24, 678.
[24] S. E. Golding, E. Rosenberg, A. Khalil, A. McEwen, M. Holmes, S. Neill, L. F. Povirk, K. Valerie, J. Biol. Chem. 2004, 279, 15402.
[25] K. Schumann, S. Lin, E. Boyer, D. R. Simeonov, M. Subramaniam, R. E. Gate, G. E. Haliburton, J. Y. Chun, J. A. Bluestone, J. A. Doudna, Proc. Natl. Acad. Sci. U.S.A. 2015, 112, 10437.

1. A composition for delivering a nucleic acid encoding an endonuclease and a nucleic acid sequence encoding a donor protein to a cell comprising:
a first plurality of self-assembled supramolecular nanoparticles (SMNPs), each of the first plurality of self-assembled supramolecular nanoparticles (SMNPs) comprising:
a plurality of binding components, each having a plurality of binding regions;
a plurality of cores that are suitable to at least provide some mechanical structure to the plurality of self-assembled supramolecular nanoparticles (SMNPs), the plurality of cores comprising at least one core binding element adapted to bind to the binding regions to form a first inclusion complex, wherein the plurality of binding components and the plurality of cores self-assemble when brought into contact to form the first plurality of self-assembled supramolecular nanoparticles (SMNPs);
a plurality of terminating components, each having a single terminating binding element that binds to remaining binding regions of one of said plurality of binding components by forming a second inclusion complex, wherein the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle;
the nucleic acid sequence encoding the endonuclease; and
a nucleotide sequence comprising a recognition sequence specific to the endonuclease; and
a second plurality of self-assembled supramolecular nanoparticles (SMNPs), each of the second plurality of self-assembled supramolecular nanoparticles (SMNPs) comprising:
a plurality of binding components, each having a plurality of binding regions;
a plurality of cores that are suitable to at least provide some mechanical structure to the plurality of self-assembled supramolecular nanoparticles (SMNPs), the plurality of cores comprising at least one core binding element adapted to bind to the binding regions to form a first inclusion complex, wherein the plurality of binding components and the plurality of cores self-assemble when brought into contact to form the second plurality of self-assembled supramolecular nanoparticles (SMNPs);
a plurality of terminating components, each having a single terminating binding element that binds to remaining binding regions of one of said plurality of binding components by forming a second inclusion complex, wherein the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle; and
the nucleic acid sequence encoding the donor protein,
wherein the nucleic acid sequence encoding the endonuclease and the nucleotide sequence comprising a recognition sequence specific to the endonuclease are encapsulated within each of the first plurality of self-assembled SMNPs, and
wherein the nucleic acid sequence encoding the donor protein is encapsulated within each of the second plurality of self-assembled SMNPs.

2. The composition of claim 1, wherein the endonuclease is a CRISPR associated protein 9 (Cas9), and
wherein the nucleotide sequence is a single guide RNA (sgRNA).

3. The composition of claim 1, wherein the plurality of cores and the plurality of binding components making up the first plurality of self-assembled SMNPs are present in a percent mass (w/w) ratio of between 0.5:1 and 3.0-1.

4. The composition of claim 1, wherein the plurality of cores and the plurality of binding components making up the second plurality of self-assembled SMNPs are present in a percent mass (w/w) ratio of between 0.5:1 and 3.0-1.

5. The composition of claim 1, wherein the plurality of terminating components of the first plurality of self-assembled SMNPs comprise a membrane penetration ligand.

6. The composition of claim 1, wherein the plurality of terminating components of the second plurality of self-assembled SMNPs comprise a membrane penetration ligand.

7. The composition of claim 1, wherein each of the first and second plurality of self-assembled supramolecular nanoparticles (SMNPs) has a diameter of between 40 nanometers and 600 nanometers.

8. The composition of claim 1, wherein the plurality of binding components of the first and second plurality of self-assembled SMNPs comprises polythylenimine, poly(L-lysine), or poly(β-amino ester).

9. The composition of claim 1, wherein the plurality of binding regions of the first and second plurality of self-assembled SMNPs comprises beta-cyclodextrin, alpha-cyclodextrin, gamma-cyclodextrin, cucurbituril or calixarene.

10. The composition of claim 1, wherein the plurality of cores of the first and second plurality of self-assembled SMNPs comprises polyamidoamine dendrimers, poly(prophylenimine) (PPI) dendrimer, triazine dendrimer, carbosilane dendrimer, poly(ether imine) (PETIM) dendrimer or phosphorus dendrimer.

11. The composition of claim 1, wherein the at least one core binding element of the first and second plurality of self-assembled SMNPs comprises adamantane, azobenzene, ferrocene or anthracene.

12. The composition of claim 1, wherein the plurality of terminating components of the first and second plurality of self-assembled SMNPs comprises polyethylene glycol (PEG) or poly(propylene glycol) (PGG).

13. The composition of claim 1, wherein the single terminating binding element of the first and second plurality of self-assembled SMNPs comprises adamantane, azobenzene, ferrocene or anthracene.

14. A method for delivering a nucleic acid encoding an endonuclease and a nucleic acid sequence encoding a donor protein to a cell comprising:
providing a first plurality of self-assembled supramolecular nanoparticles (SMNPs);
providing a second plurality of self-assembled SMNPs; and
contacting the cell with at least one of the first plurality of self-assembled SMNPs and with at least one of the second plurality of self-assembled SMNPs, such that the at least one of the first plurality of self-assembled SMNPs and the at least one of the second plurality of self-assembled SMNPs are each taken up by the cell,
wherein each of the first plurality of self-assembled supramolecular nanoparticles SMNPs comprises:
a plurality of binding components, each having a plurality of binding regions;
a plurality of cores that are suitable to at least provide some mechanical structure to the plurality of self-assembled SMNPs, the plurality of cores comprising at least one core binding element adapted to bind to the binding regions to form a first inclusion complex, wherein the plurality of binding components and the plurality of cores self-assemble when brought into contact to form the first plurality of self-assembled SMNPs;
a plurality of terminating components, each having a single terminating binding element that binds to remaining binding regions of one of said plurality of binding components by forming a second inclusion complex, wherein the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle;
the nucleic acid sequence encoding the endonuclease; and
a nucleotide sequence comprising a recognition sequence specific to the endonuclease, wherein each of the second plurality of self-assembled SMNPs comprises:
a plurality of binding components, each having a plurality of binding regions;
a plurality of cores that are suitable to at least provide some mechanical structure to the plurality of self-assembled SMNPs, the plurality of cores comprising at least one core binding element adapted to bind to the binding regions to form a first inclusion complex, wherein the plurality of binding components and the plurality of cores self-assemble when brought into contact to form the second plurality of self-assembled SMNPs;
a plurality of terminating components, each having a single terminating binding element that binds to remaining binding regions of one of said plurality of binding components by forming a second inclusion complex, wherein the plurality of terminating components act to occupy the remaining binding regions of the plurality of binding components, and the plurality of terminating components are present in a sufficient quantity relative to the plurality of binding regions of the plurality of binding components to terminate further binding, thereby forming a discrete particle; and
the nucleic acid sequence encoding the donor protein,
wherein the nucleic acid sequence encoding the endonuclease and the nucleotide sequence comprising a recognition sequence specific to the endonuclease are encapsulated within each of the first plurality of self-assembled SMNPs, and
wherein the nucleic acid sequence encoding the donor protein is encapsulated within each of the second plurality of self-assembled SMNPs.

15. The method of claim 14, wherein the endonuclease is a CRISPR associated protein 9 (Cas9), and
wherein the nucleotide sequence is a single guide RNA (sgRNA).

16. The method of claim 14, wherein the plurality of cores and the plurality of binding components making up the first plurality of self-assembled SMNPs are present in a percent mass (w/w) ratio of between 0.5:1 and 3.0-1.

17. The method of claim 14, wherein the plurality of cores and the plurality of binding components making up the second plurality of self-assembled SMNPs are present in a percent mass (w/w) ratio of between 0.5:1 and 3.0-1.

18. The method of claim 14, wherein the plurality of terminating components of the first plurality of self-assembled SMNPs comprise a membrane penetration ligand.

19. The method of claim 14, wherein the plurality of terminating components of the second plurality of self-assembled SMNPs comprise a membrane penetration ligand.

20. The method of claim 14, wherein each of the first and second plurality of self-assembled supramolecular nanoparticles (SMNPs) has a diameter of between 40 nanometers and 600 nanometers.

21. The method of claim 14, wherein the plurality of binding components of the first and second plurality of self-assembled SMNPs comprises polythylenimine, poly(L-lysine), or poly(β-amino ester).

22. The method of claim 14, wherein the plurality of binding regions of the first and second plurality of self-assembled SMNPs comprises beta-cyclodextrin, alpha-cyclodextrin, gamma-cyclodextrin, cucurbituril or calixarene.

23. The method of claim 14, wherein the plurality of cores of the first and second plurality of self-assembled SMNPs comprises polyamidoamine dendrimers, poly(prophylenimine) (PPI) dendrimer, triazine dendrimer, carbosilane dendrimer, poly(ether imine) (PETIM) dendrimer or phosphorus dendrimer.

24. The method of claim 14, wherein the at least one core binding element of the first and second plurality of self-assembled SMNPs comprises adamantane, azobenzene, ferrocene or anthracene.

25. The method of claim 14, wherein the plurality of terminating components of the first and second plurality of self-assembled SMNPs comprises polyethylene glycol (PEG) or poly(propylene glycol) (PGG).

26. The method of claim 14, wherein the single terminating binding element of the first and second plurality of self-assembled SMNPs comprises adamantane, azobenzene, ferrocene or anthracene.
